(19) United States
(12) Patent Application Publication
(10) Pub. No.: US 2026/0283478 A1
(43) Pub. Date: Sep. 24, 2026

(54) ASEPTIC NON-INVASIVE VALSALVA MANEUVER APPARATUS AND METHOD FOR ENHANCING ECHOCARDIOGRAPHIC DIAGNOSIS

(71) Applicants: Sahaj Anilbhai Patel, Hoover, AL (US); Garima Arora, Birmingham, AL (US)

(72) Inventors: Sahaj Anilbhai Patel, Hoover, AL (US); Garima Arora, Birmingham, AL (US)

(21) Appl. No.: 19/374,181

(22) Filed: Oct. 30, 2025

(51) Int. Cl. A61B 5/02; A61B 5/00; A61B 8/06; A61B 8/08; A61B 90/00

(52) U.S. Cl. CPC A61B 5/02028; A61B 5/742; A61B 5/7475; A61B 90/08; A61B 8/065; A61B 8/0883; A61B 2090/0811; A61B 2090/0814; A61B 2560/0214

(57) ABSTRACT

An aseptic, non-invasive Valsalva Maneuver (VM) apparatus and method are presented for enhancing echocardiographic diagnoses performed by sonographers. The apparatus is designed to improve evaluation and optimize the assessment of cardiovascular conditions such as Hypertrophic Cardiomyopathy (HCM), Patent Foramen Ovale (PFO), and Diastolic Dysfunction, where precise diagnostic measurements are critical for clinical management and treatment decisions. The device comprises two modules: a top module equipped with a digital display and a controller for real-time pressure data processing, and a bottom module housing a pressure transducer. A flexible tube with a mouthpiece is connected to the transducer, enabling the patient to perform the VM. The modules are magnetically coupled, with connector pins in both modules enabling reliable data transfer. For infection control, the bottom module, along with the flexible tube and mouthpiece, is designed for single-patient use and is disposable after each procedure, thereby maintaining aseptic conditions and preventing cross-contamination.

### BACKGROUND OF THE INVENTION

The term “Valsalva Maneuver (VM)” was first introduced in 1704 by Antonio Valsalva, a renowned Italian anatomist.¹ VM is a breathing technique in which an individual exhales forcefully against a closed airway by closing their mouth and pinching their nose shut. The first clinical study of VM's physiological effects on the cardiovascular system was conducted in 1850 by Ernst Heinrich Weber.² Weber observed that by closing the airways using the VM technique, there was a noticeable change in pressure between the chest and the circulatory system. These changes in effects due to VM are called hemodynamic effects. These hemodynamic responses, triggered by VM, enhance the diagnostic capability for complex conditions such as Hypertrophic Cardiomyopathy (HCM), Patent Foramen Ovale (PFO), and diastolic dysfunction during echocardiography examination. Over time, VM has become an integral tool in cardiovascular medicine for unmasking the various abnormalities in cardiac function during echocardiography.

The hemodynamic effects of the VM generally include four distinct phases, typically spanning a total duration of 10-20 seconds. In Phase 1 (Initial phase), lasting a few initial seconds (1-3 seconds), there is a rise in blood pressure and reduction in heart rate due to increased intrathoracic pressure and outflow of blood from the left atrium. Phase 2 (Strain phase) generally lasts between 5-14 seconds, during which the blood pressure and heart rate remain low overall. In the early seconds of Phase 2, there is a reduction in venous return to the heart, decreased stroke volume, and reduced cardiac output, leading to a reduction in blood pressure. In late Phase 2, baroreceptors detect the reduced blood pressure, activating the sympathetic nervous system to gradually increase heart rate, and blood pressure starts to recover to near or above normal levels. Phase 3 (Release phase) lasts 1-2 seconds and is characterized by a sudden drop in blood pressure due to a rapid decrease in intrathoracic pressure. In Phase 4 (Overshoot phase), venous return normalizes, resulting in rapid outflow of blood from heart, which overshoots the blood pressure above baseline levels before returning to normal.³ Each phase is utilized by sonographers for diagnosing different cardiovascular conditions.

For instance, Phase 2 can help evaluate various properties of HCM and Diastolic dysfunction.⁴ HCM, the most common inherited cardiac condition, is characterized by abnormal thickening of the heart muscle, which may result in impaired cardiac function, arrhythmias, and an increased risk of sudden cardiac death, particularly in young, otherwise healthy individuals. Accurate assessment of left ventricular outflow tract (LVOT) gradients is critical for managing obstructive HCM, especially in patients being considered for Mavacamten, a myosin inhibitor that has demonstrated significant benefits in reducing obstruction and alleviating symptoms.^{5,6}

Diastolic dysfunction, on the other hand, refers to the inability of the left ventricle to relax properly during diastole, leading to stiffness, impaired filling, and increased intracardiac pressure. It is a major contributor to heart failure with preserved ejection fraction (HFpEF), a condition that is becoming increasingly prevalent in aging populations. If left undetected and untreated, diastolic dysfunction can progress to overt heart failure, underscoring the importance of early diagnosis and management.⁷

In addition to assessing diastolic dysfunction, Phase 3 of the VM provides valuable insights into PFO.⁴ PFO is a common congenital heart defect where a hole between the heart's atria fails to close after birth. While often asymptomatic, PFO is of significant clinical relevance in young patients with cryptogenic stroke, where the cause of the stroke remains unexplained despite extensive testing. In these cases, PFO closure with an occluder device is an important intervention to prevent recurrent strokes.⁸⁻¹⁰ The success of PFO occlusion is heavily dependent on the accurate measurement of the shunt size, which can be difficult to assess without proper stress testing. The VM induces transient pressure changes that can increase the size of the shunt, helping to reveal its true dimensions during echocardiography.

Traditionally, the VM performed during echocardiography is carried out using a mechanical manometer.^{4,11} Patients are instructed to maintain air pressure above a specified threshold, typically 40 mmHg, as guided by the sonographer during the examination.¹² However, many patients struggle to interpret the manometer readings, as the scales are not always fully numerical. This highlights a critical need for a digital VM device that offers improved readability, ensuring usability regardless of the patient's educational background.

Moreover, the lack of a standardized VM device capable of delivering accurate and reproducible results further underscores the necessity for innovation. A standardized digital VM device would ensure reliable execution of the maneuver, allowing for precise measurements of shunt size and enabling clinicians to make better-informed decisions regarding the need for PFO closure to prevent recurrent strokes.

Besides PFO evaluation, the accuracy of LVOT gradient measurements is significant for therapies such as Mavacamten, which require precise assessment of obstruction severity to determine appropriate dosing.⁵ The VM is integral to this process, as it dynamically alters left ventricular pressure and flow, providing a dependable method for evaluating obstruction severity. A standardized VM device would guarantee accurate and reproducible gradient measurements, which are essential for optimal therapy management and improved patient outcomes in obstructive HCM.

Beyond to the readability issues of the manometer and reproducible result of VM device, a digital VM device would allow sonographers to accurately assess changes in patient symptoms with changes in applied pressure. Additionally, by facilitating accurate diagnosis and determining the most effective therapy, the device can help reduce the financial burden on both patients and healthcare systems by minimizing unnecessary diagnostic tests, such as cardiac MRI (Magnetic Resonance Imaging). According to a report by the HCM Association in 2000, 1 in 500 people are affected by HCM, which sums up to between 700,000 to 725,000 cases in the United States. The HCM-related cost alone can be about $5,968 at baseline with $20,290 per patient for a 1-year follow-up.¹³ Furthermore, a digital VM can help reduce invasive procedures such as supraventricular tachycardia (SVT) by simulating stress conditions.

In addition to the financial implications, an equally important concern is the risk of cross-contamination and infection, regardless of whether a digital or mechanical manometer is used. While the mouthpiece and tubing are typically discarded after each examination, the sensor unit is often reused across multiple patients. Following the COVID-19 pandemic, numerous studies have shown how air contamination or surface-to-surface contact can spread viruses easily between patients. Therefore, there is a critical need for a disinfected device to ensure patient safety during echocardiographic examinations involving the VM.

### REFERENCES

1. Jellinek E H. The Valsalva manoeuvre and Antonio Valsalva (1666-1723). J R Soc Med. 2006; 99:448-451. doi: 10.1177/014107680609900915
2. Jellinek E H. The Valsalva manoeuvre and Antonio Valsalva (1666-1723). Journal of the Royal Society of Medicine. 2006; 99:448-451. doi: 10.1177/014107680609900915
3. McGee S. Evidence-based physical diagnosis. Philadelphia, PA: W. B. Saunders; 2001.
4. Zhao E, Zhang Y, Kang C, Niu H, Zhao J, Sun L, Liu B. Influence of the Valsalva maneuver on cardiac hemodynamics and right to left shunt in patients with patent foramen ovale. Sci Rep. 2017; 7:44280. doi: 10.1038/srep44280
5. Olivotto I, Oreziak A, Barriales-Villa R, Abraham T P, Masri A, Garcia-Pavia P, Saberi S, Lakdawala N K, Wheeler M T, Owens A, et al. Mavacamten for treatment of symptomatic obstructive hypertrophic cardiomyopathy (EXPLORER-HCM): a randomised, double-blind, placebo-controlled, phase 3 trial. The Lancet. 2020; 396:759-769. doi: 10.1016/S0140-6736(20)31792-X
6. Ommen S R, Ho C Y, Asif I M, Balaji S, Burke M A, Day S M, Dearani J A, Epps K C, Evanovich L, Ferrari V A, et al. 2024 AHA/ACC/AMSSM/HRS/PACES/SCMR Guideline for the Management of Hypertrophic Cardiomyopathy: A Report of the American Heart Association/American College of Cardiology Joint Committee on Clinical Practice Guidelines. Circulation. 2024; 149:e1239-e1311. doi: doi:10.1161/CIR.0000000000001250
7. Obokata M, Reddy Y N V, Borlaug B A. Diastolic Dysfunction and Heart Failure With Preserved Ejection Fraction. JACC: Cardiovascular Imaging. 2020; 13:245-257. doi: doi: 10.1016/j.jcmg.2018.12.034
8. Søndergaard L, Kasner S E, Rhodes J F, Andersen G, Iversen H K, Nielsen-Kudsk J E, Settergren M, Sjöstrand C, Roine R O, Hildick-Smith D, et al. Patent Foramen Ovale Closure or Antiplatelet Therapy for Cryptogenic Stroke. New England Journal of Medicine. 2017; 377:1033-1042. doi: doi: 10.1056/NEJMoa 1707404
9. Saver J L, Carroll J D, Thaler D E, Smalling R W, MacDonald L A, Marks D S, Tirschwell D L. Long-Term Outcomes of Patent Foramen Ovale Closure or Medical Therapy after Stroke. New England Journal of Medicine. 2017; 377:1022-1032. doi: doi: 10.1056/NEJMoa1610057
10. Mas J-L, Derumeaux G, Guillon B, Massardier E, Hosseini H, Mechtouff L, Arquizan C, Béjot Y, Vuillier F, Detante O, et al. Patent Foramen Ovale Closure or Anticoagulation vs. Antiplatelets after Stroke. New England Journal of Medicine. 2017; 377:1011-1021. doi: doi:10.1056/NEJMoa1705915
11. Hotta V T, Romano M M D, Barberato S H, Vieira M L C, Fernandes F, Simões M V. The Importance of the Effective Valsalva Maneuver during Echocardiography in Hypertrophic Cardiomyopathy. Arquivos Brasileiros de Cardiologia. 2024; 121:-. doi: 10.36660/abc.20230871i
12. Kumar S, Van Ness G, Bender A, Yadava M, Minnier J, Ravi S, McGrath L, Song H K, Heitner S B. Standardized Goal-Directed Valsalva Maneuver for Assessment of Inducible Left Ventricular Outflow Tract Obstruction in Hypertrophic Cardiomyopathy. Journal of the American Society of Echocardiography. 2018; 31:791-798. doi: https://doi.org/10.1016/j.echo.2018.01.022
13. Butzner M, Maron M, Sarocco P, Teng C C, Stanek E, Tan H, Robertson L. Healthcare resource utilization and cost of obstructive hypertrophic cardiomyopathy in a US population. Am Heart J Plus. 2022; 13:100089. doi: 10.1016/j.ahjo.2022.100089

### BRIEF SUMMARY OF THE INVENTION

The present invention introduces a digital VM device designed to overcome the limitations of traditional mechanical manometers while enhancing patient engagement and diagnostic precision. The invention improves patient readability and supports precise gradient measurements, which are critical for managing HCM and Mavacamten therapy. Additionally, the device enables accurate assessment of shunt size in cases of PFO and enhances diagnostic accuracy in evaluating diastolic dysfunction.

The digital VM device comprises two main modules: a reusable top module and a disposable bottom module. The top module includes a casing that houses a digital display configured to provide real-time pressure readings in mmHg, elapsed time, and a customizable digital scale with a marker set by the sonographer. This feature enables patients to easily interpret and maintain the required pressure threshold during the examination, regardless of their educational background. The top module further comprises a microprocessor, a power push button, and a battery. Additionally, the top module includes a pin connector located at its bottom surface, which facilitate the transfer and reading of airflow pressure signals transmitted from the bottom module. The top module also incorporates mounting notches that allow it to be secured to any universal stand or fixed directly onto an echocardiogram machine, thereby eliminating the need for the patient or sonographer to manually hold the unit during the digital VM examination.

The bottom module consists of a casing that houses a pressure transducer and a connector with spring pins for transmitting pressure data to the top module. The pressure transducer tip is attached to a flexible tube and mouthpiece. The top and bottom modules are magnetically coupled, ensuring secure and reliable communication between them. After each use, the bottom module can be detached and safely disposed of, thereby preventing cross-contamination and infection between patients.

In summary, the present digital VM device modernizes traditional VM techniques by enhancing patient comprehension and improving diagnostic efficiency in the evaluation of HCM, PFO, and diastolic dysfunction. The device further reduces healthcare costs while maintaining high standards of hygiene and safety. In addition, its modular and adaptable design supports expanded applications in the assessment of other cardiovascular conditions that require dynamic stress testing to guide effective treatment strategies.

### BRIEF DESCRIPTION OF THE DRAWINGS

FIG. 1. Illustrates a perspective view of a clinical environment in which a patient is using the assembled digital VM device during a cardiac examination performed with an echocardiographic machine by a sonographer.

FIG. 2A. Provides a detailed, disassembled view of the top module of the digital VM device, highlighting its internal components.

FIG. 2B. Shows the mounting hole for the enclosure plate and the alignment marker in the top module of the digital VM device.

FIG. 2C. Illustrates the locations of the power push button in the top module of the digital VM device.

FIG. 2D. Illustrates the location of the charging port in the top module of the digital VM device.

FIG. 2E. Demonstrates the mounting notches and their associated grippers. The grippers are configured to fit onto a universal stand or an echocardiogram machine for securely holding the top module of the digital VM device, along with the bottom sensor module when magnetically attached to the top module.

FIG. 3A. Presents a detailed, disassembled view of the bottom module of the digital VM device, showcasing its internal components, including the alignment marker for user guidance on the enclosure plate, as well as the locations of the magnets that are inserted into the enclosure plate of the bottom module.

FIG. 3B. Provides a perspective view of the pressure sensor connection with the mouthpiece without the bottom module casing. It shows the connection of the flexible tube to the tip of pressure transducer and the flexible tube with an elliptical shaped mouthpiece designed for an airtight fit with the patient's mouth.

### DETAILED DESCRIPTION OF THE INVENTION

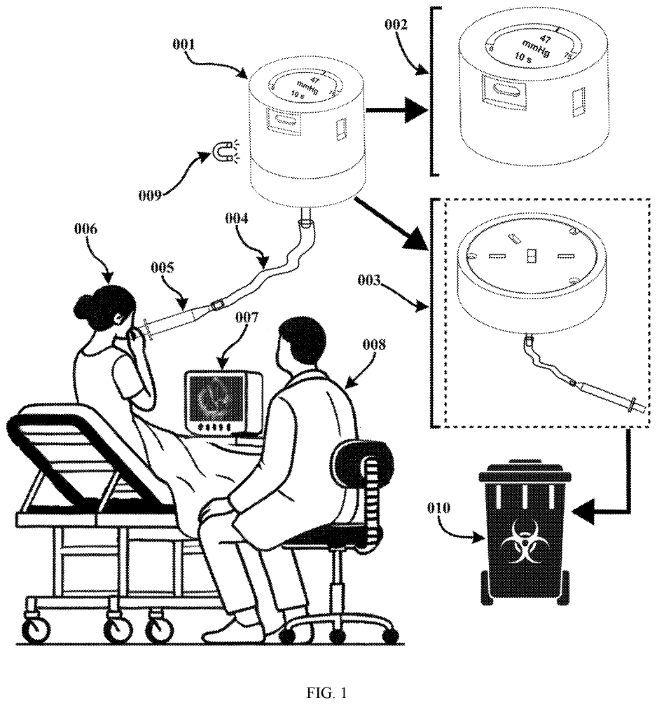

FIG. 1 illustrates an embodiment of the present invention, depicting the overall clinical environment in which the fully assembled digital VM device 001 is utilized by a patient 006. An echocardiographic diagnosis is performed by a sonographer 008, who visualizes the patient's echocardiography on a machine screen 007 while instructing the patient 006 to blow air into the device's mouthpiece 005. The mouthpiece 005 is connected to the digital VM device 001 via a flexible tube 004. The digital VM device 001 comprises a top assembled module 002 and a bottom assembled module 003, together with the flexible tube 004 and mouthpiece 005. Both modules 002 and 003 are magnetically coupled by a magnetic force 009 generated by magnets positioned within the assembled modules. After completion of the diagnostic procedure, the sonographer 008 disposes of the entire bottom module 003, including the flexible tube 004 and mouthpiece 005, into a biohazard trash bin 010, thereby preventing cross-contamination and infection during subsequent patient examinations.

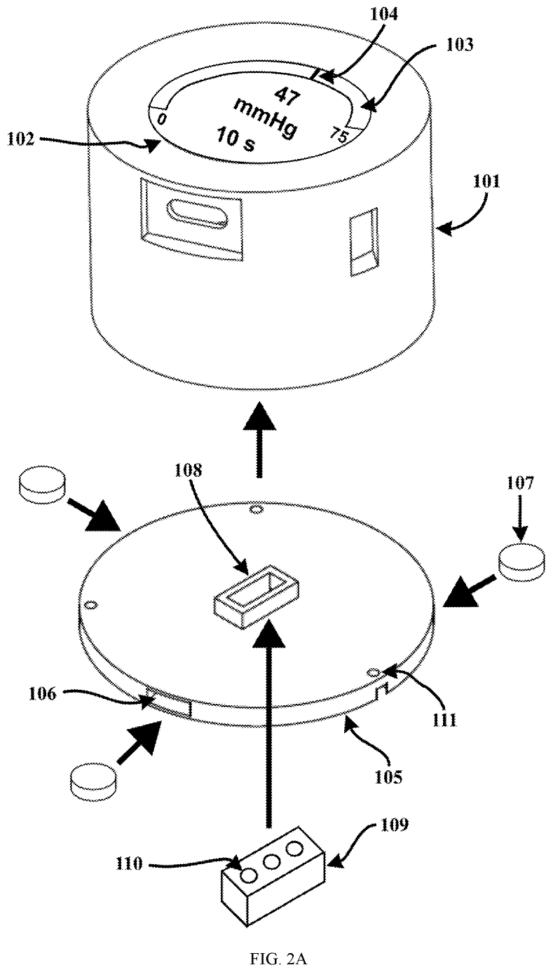

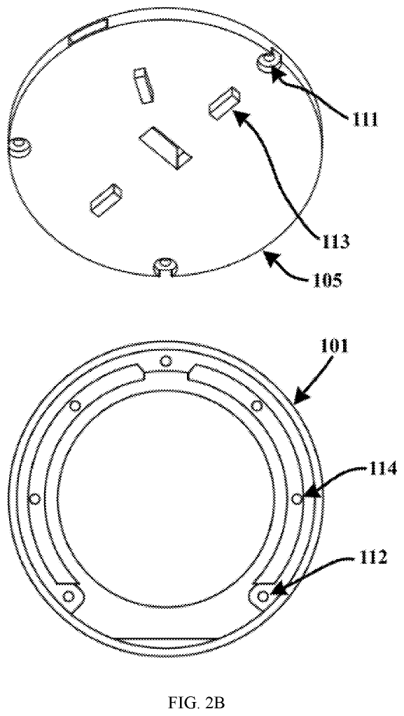

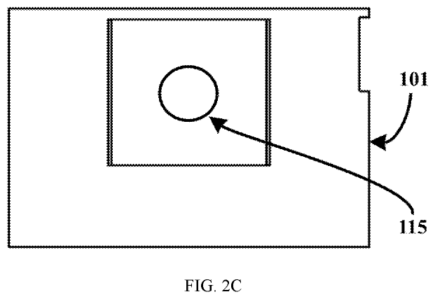

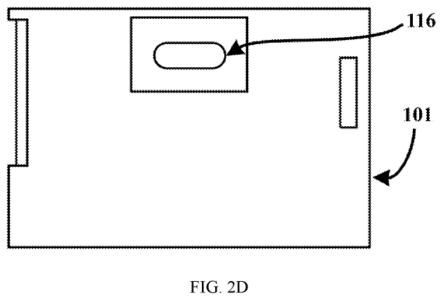

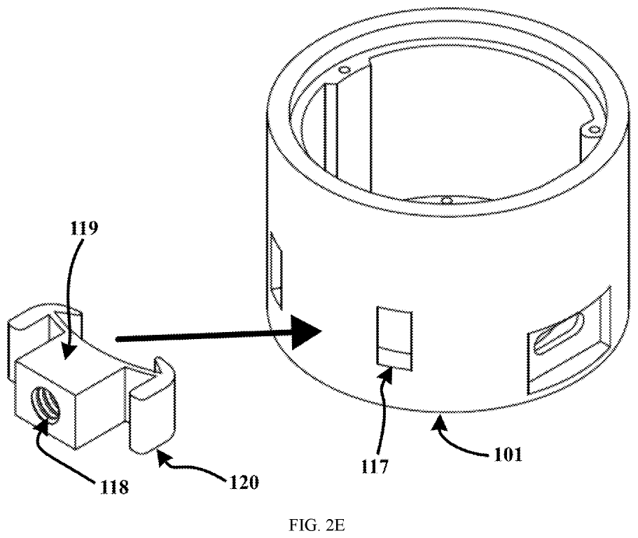

FIG. 2A depicts the detailed internal design and components of the assembled top module 002. The top module 002 comprises a hard casing 101, which protects the internal components from environmental wear and tear. The casing 101 houses a digital display 102 configured for easy readability by both the patient 006 and the sonographer 008. The display 102 indicates the pressure in mmHg exerted by the patient 006 through the mouthpiece 005, and also displays the elapsed time in seconds from the initiation of pressure exertion until the resting mode. To enhance patient comprehension, the display 102 incorporates a digital arc-shaped scale 103, functionally similar to a mechanical manometer. Each increment on the scale 103 corresponds to 1 mmHg, beginning at zero on the left side and extending to a maximum of 75 mmHg on the right side, consistent with the capacity of the pressure transducer. A customized marker 104 may be set on the scale 103 according to the sonographer's 008 desired pressure requirement during examination, thereby engaging the patient 006 to maintain pressure above the marker. The top assembled module 002 further comprises a processor, a battery, and an enclosure plate 105 for the casing 101. The enclosure plate 105 contains slots 106 for receiving magnets 107, and a slot 108 for a pin connector 109. The connector 109 includes conductive pins 110 that enable communication between the top module 002 and the bottom module 003 of the VM device.

FIG. 2B illustrates the screw holes configured for securing the top module casing 101 to its enclosure plate 105, along with a rectangular-shaped protruding alignment marker 113. The alignment marker 113 assists the sonographer 008 in properly aligning the top module 002 with the bottom module 003 when magnetically coupling the two modules. The casing 101 of the top module 002 is enclosed and protected by the enclosure plate 105, which is secured using screws inserted through screw holes 111 and 112, thereby safeguarding the internal components from harsh external conditions.

FIG. 2C represents the ON/OFF power push button 115 of the VM device 001. The push button 115 is mounted on the outer surface of the top module casing 101 and is configured to control the power supply to the device. This allows the sonographer 008 to conveniently switch the device between operational and standby modes during the examination without interrupting patient use.

FIG. 2D illustrates the charging port 116, which is integrated into the top module casing 101. The charging port 116 allows recharging of the internal battery of the top module through an external power source, thereby enabling repeated use of the reusable module. Placement of the charging port 116 on the outer surface of the casing 101 ensures easy accessibility for the operator while preserving the structural integrity and protective function of the casing against environmental factors.

FIG. 2E shows two mounting slots 117 provided on the top module 101. These slots 117 are designed to enable hands-free usage of the VM device 001. A gripper 119 is configured to be inserted into the slots 117 by pressing its two ends 120. The gripper 119 includes a threaded portion 118, which facilitates secure mounting of the VM device 001 onto a universal camera tripod, a mobile phone holder stand, or a stand integrated with an echocardiogram machine. This configuration allows the VM device 001 to remain in a stable position during operation without requiring the patient or sonographer to hold the device manually.

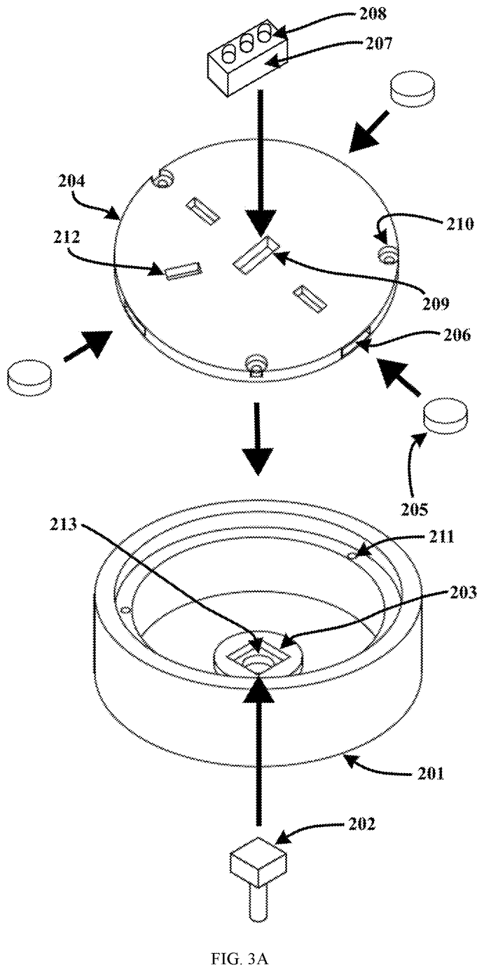

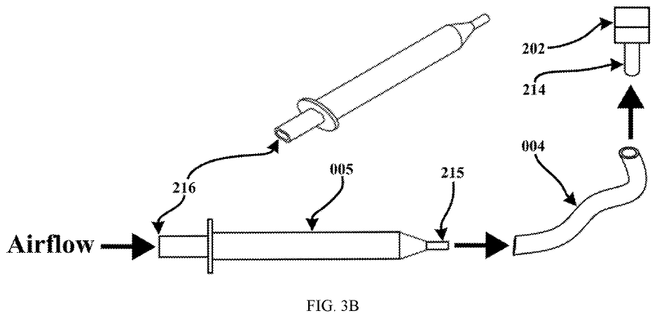

FIG. 3A shows a detailed, disassembled view of all components within the bottom module 003 of the VM device 001. Similar to the top module 002, the bottom module comprises a casing 201 and an enclosure plate 204. The enclosure plate 204 includes slots 206 for accommodating magnets 205, oriented with opposite polarity to magnets 107 of the top module, thereby enabling secure magnetic coupling between the two modules. The enclosure plate 204 further includes a slot for receiving a connector 207 equipped with conductive spring pins 208. The connector 207, which is secured in a slot 209 of the enclosure plate 204, is configured to transmit pressure data from the bottom module 003 to the top module 002.

The enclosure plate 204 also comprises alignment markers 212, corresponding in number to the alignment markers 113 of the top module. Unlike the raised alignment markers 113, the markers 212 on the enclosure plate 204 are recessed, enabling the two sets of markers to interlock when aligned. This prevents relative rotation between the top module 002 and the bottom module 003, thereby ensuring stable positioning during use. The bottom casing 201 further includes a slot 203 for receiving the pressure transducer 202, securing it firmly in place. The tip 214 of the pressure transducer 202 extends outward through an outlet hole 213 in the bottom module, as shown in FIG. 3B. All internal components of the bottom module 003 are enclosed and protected by the enclosure plate 204, which is fastened to the casing 201 by screws inserted through holes 210 and 211.

FIG. 3B provides a perspective view of the connection between the mouthpiece 005 and the flexible tube 004, which in turn connects to the pressure transducer tip 214 extending through the outlet hole 213 of the bottom module 003. One end of the flexible tube 004 is securely fitted onto the pressure transducer tip 214 without requiring additional tightening mechanisms such as clamps, clips, or zip ties. The opposite end of the flexible tube 004 is connected to the circular tip 215 of the mouthpiece 005. The patient introduces airflow into the system by blowing through the distal end 216 of the mouthpiece 005. The distal end 216 is ergonomically designed to conform to the contours of the patient's mouth, thereby minimizing air leakage from the sides and ensuring accurate pressure transmission to the transducer 202.

1. An apparatus for performing a Valsalva Maneuver during echocardiographic diagnosis, comprising: a reusable top module comprising a casing, a digital display, a microprocessor, a rechargeable power supply, and a first connector with conductive pins; a disposable bottom module comprising a casing, a pressure transducer, a second connector with spring-loaded pins configured to engage the first connector, a flexible tube, and a mouthpiece; and a magnetic coupling mechanism configured to releasably secure the top and bottom modules; wherein the digital display presents in real time pressure values in millimeters of mercury (mmHg), elapsed time, and a clinician-set threshold marker, and wherein the bottom module, flexible tube, and mouthpiece are configured for single-patient use and disposal.

2. A system for facilitating echocardiographic diagnosis, comprising: the apparatus of claim 1; and an echocardiography machine positioned for concurrent imaging, wherein the apparatus provides real-time pressure feedback to a patient and sonographer while the echocardiography machine captures cardiac images.

3. A method of performing a Valsalva Maneuver during echocardiographic diagnosis, comprising: coupling a reusable top module with a disposable bottom module; instructing a patient to exhale into a mouthpiece connected to the pressure transducer of the bottom module; generating pressure signals with the transducer and processing the signals in the top module; displaying in real time pressure values, elapsed time, and a clinician-set threshold on a digital display; and disposing of the bottom module, flexible tube, and mouthpiece after use.

4. A method of reducing cross-contamination during repeated diagnostic examinations, comprising: confining patient-contact airflow components within a disposable module, maintaining electronic processing components within a reusable module, and discarding the disposable module after each patient procedure.

5. The apparatus of claim 1, wherein the digital display comprises an arc-shaped scale with increments of 1 mmHg up to 75 mmHg.

6. The apparatus of claim 1, wherein the clinician-set threshold marker is movable along the digital scale.

7. The apparatus of claim 1, wherein the casing of the top module comprises mounting notches configured to engage a gripper for attachment to a stand or echocardiography machine.

8. The apparatus of claim 1, wherein the rechargeable power supply comprises a lithium-ion battery accessible through an external charging port.

9. The apparatus of claim 1, wherein the magnetic coupling mechanism comprises rare-earth magnets embedded in enclosure plates of the respective modules.

10. The apparatus of claim 1, wherein the top and bottom modules include complementary alignment markers configured to prevent relative rotation.

11. The apparatus of claim 1, wherein the mouthpiece comprises an elliptical distal end configured to minimize leakage at the lips.

12. The apparatus of claim 1, wherein the digital display further includes an alert indicator operative to notify a patient when pressure falls below the threshold.

13. The method of claim 3, wherein maintaining patient pressure above the threshold for a predetermined duration enables assessment of left ventricular outflow tract gradients in hypertrophic cardiomyopathy.

14. The method of claim 3, wherein release of pressure during the maneuver facilitates visualization of right-to-left shunting across a patent foramen ovale.

15. The method of claim 3, wherein changes in transmitral filling velocities during the maneuver assist in grading diastolic dysfunction.

16. The method of claim 3, further comprising adjusting the threshold marker to correspond with a hypertrophic cardiomyopathy treatment protocol.

17. The method of claim 3, further comprising storing pressure values and elapsed time in association with a patient record.

18. The method of claim 3, wherein the top module is secured to a stand prior to use to allow hands-free operation.

19. The method of claim 4, wherein the discarded disposable module is placed in a biohazard receptacle.

20. The method of claim 3, wherein the sonographer references the displayed pressure values simultaneously with echocardiographic imaging for diagnostic interpretation.
